(19) United States
(12) Patent Application Publication
(10) Pub. No.: US 2022/0184084 A1
(43) Pub. Date: Jun. 16, 2022

(54) COMPOSITIONS AND METHODS FOR TREATING CUSHING'S DISEASE

(71) Applicant: The Regents of the University of California, Oakland, CA (US)

(72) Inventors: Anthony Heaney, Los Angeles, CA (US); Dongyun Zhang, Los Angeles, CA (US); Robert Damoiseaux, Los Angeles, CA (US)

(21) Appl. No.: 17/440,591

(22) PCT Filed: Mar. 18, 2020
(86) PCT No.: PCT/US20/23265

§ 371 Date: Sep. 17, 2021

Related U.S. Application Data
(60) Provisional application No. 62/820,059, filed on Mar. 18, 2019.

(51) Int. Cl. A61K 31/519; A61K 31/454; A61K 31/496; A61K 31/5377; A61P 35/00; A61P 5/08

(52) U.S. Cl. CPC A61K 31/519; A61K 31/454; A61P 5/08; A61K 31/5377; A61P 35/00; A61K 31/496

(57) ABSTRACT

Disclosed herein are methods of treating a disease or disorder characterized by an increased secretion of adrenocorticotropic hormone (e.g., Cushing's disease). Also disclosed herein are methods of identifying compounds for use in treating said diseases or disorders.

### RELATED APPLICATIONS

This application claims the benefit of U.S. Provisional Application No. 62/820,059, filed Mar. 18, 2019, the contents of which are fully incorporated by reference herein.

### BACKGROUND

Cushing Disease (CD) is a life-threatening “orphan disease” with an annual US incidence of ˜8 cases per million (Broder et al. 2015). It is caused by an adrenocorticotropic hormone (ACTH)-secreting pituitary adenoma, which drives excess adrenal-derived cortisol production. CD patients have 5× the rate of osteoporosis and diabetes, 4× the rate of cardiovascular disease, liver disease, and obesity, 3× the rate of hypertension and mood disorders, and 2× the rate of dyslipidemia, menstrual abnormalities, and acne. Although initial remission rates after surgical corticotroph tumor removal in expert centers are ˜80% for microadenomas (<1 CM diameter and most common), disease recurrence is high (30-40%) and remission is very rare with larger tumors. Thereafter, therapies include repeat pituitary surgery with very poor success rates (<50%) or pituitary directed radiation therapy that takes several years to offer biochemical control and causes hypopituitarism in ˜40% of patients. Alternatively, bilateral adrenalectomy resolves hypercortisolism, but requires lifelong gluco- and mineralo-corticoid replacement and may spur rapid pituitary tumor growth in 25% of patients.

As the cause of CD is an ACTH-secreting pituitary tumor, an ideal pharmaceutical would: i) act on the tumor itself (e.g., by inhibiting growth); and ii) potently and selectively inhibit corticotroph tumor-derived ACTH at any level (e.g., transcription, post-translational prohormone processing, protein transport, and secretion) to attain sustained eucorticolism and inhibit corticotroph tumor growth. The pharmaceutical's side effect profile must be acceptable for the duration of therapy which, if the drug eradicates the tumor, could be as long as 12-24 months or potentially be lifelong.

Most currently available drugs are either adrenal-directed inhibitors of glucocorticoid synthesis (e.g., Ketoconazole or Metyrapone), or block glucocorticoid action (e.g., Korlym). Long-term compliance with these drugs is low (i.e., <30%) either due to loss of control caused by increased tumor-derived ACTH or side effects. Another example, pasireotide, is tumor-directed, does not inhibit tumor growth, and is rarely used as it causes diabetes in approximately 50% of patients. Furthermore, the annual health care cost of CD patients is >7 times higher than average patients, and there is a large unmet medical need in treatment for this “orphan disease”. Thus, there remains a clinical need for the treatment of Cushing's Disease.

### SUMMARY OF THE INVENTION

In certain aspects, the present disclosure provides a disease or disorder characterized by an increased secretion of adrenocorticotropic hormone (e.g., Cushing's disease), comprising administering to a subject in need thereof a compound that inhibits both the secretion of adrenocorticotropic hormone and tumor growth.

In certain aspects, the present disclosure provides methods of identifying a compound that inhibits both the secretion of adrenocorticotropic hormone and tumor growth.

### BRIEF DESCRIPTION OF THE DRAWINGS

FIG. 1A depicts the screen of a kinase inhibitor library (KIL) using ACTH AlphaLISA in combination with nuclei staining (Hoechst 33342 dye). Depiction of KIL compound composition (n=430).

FIG. 1B depicts AtT20 cells were treated with KIL compounds at 100 nm, 1 mM and 10 mM final concentrations. Plotted values corresponding to ACTH secretion and proliferation inhibition rates were calculated from AlphaLISA signals. The compounds exhibiting >50% ACTH inhibition (n=6, 20 and 115) were highlighted in grey for doses of 100 nm, 1 mM and 10 mM respectively.

FIG. 1C depicts AtT20 cells were treated with KIL compounds at 100 nm, 1 mM and 10 mM final concentrations. Plotted values corresponding to ACTH secretion and proliferation inhibition rates were calculated from Hoechst 33342 staining. The compounds exhibiting >50% proliferation inhibition (n=36, 105 and 263) were highlighted in grey for doses of 100 nm, 1 mM and 10 mM respectively.

FIG. 1D depicts the correlation of ACTH and nuclei inhibition; the compounds exhibited >50% inhibition in both parameters were highlighted in grey.

FIG. 2 depicts the determination of the IC₅₀ of CUDC-907, PF-3758309, Dinaciclib, NVP-BGT226, BI-2536, and PHA-793887 against AtT20 cells that were treated at 20 concentrations from 10 μM to 40 μM (2-fold dilution). The IC₅₀ values were calculated using sigmoidal dose-response curve (GraphPad Prism).

FIG. 3A depicts the effects of CUDC-907 on ACTH secretion in AtT20 cells. AtT20 cells were treated with CUDC-907 at the indicated doses for 24 h. The POMC mRNA expression and ACTH secretion values were determined by real time PCR.

FIG. 3B depicts the effects of CUDC-907 on ACTH secretion in AtT20 cells. AtT20 cells were treated with CUDC-907 at the indicated doses for 24 h and the POMC mRNA expression and ACTH secretion values were determined by real time ELISA.

FIG. 3C depicts the effects of CUDC-907 in human corticotroph tumor primary cultures (n=2). Two human corticotroph tumor primary cultures were treated with CUDC-907 for 3 days and the cell proliferation rates were determined by CellTiter-Glo to calculated and IC₅₀ value using sigmoidal dose-response curve.

FIG. 3D depicts the effects of CUDC-907 on POMC mRNA expression in corticotroph tumor primary cultures. The effects of CUDC-907 on POMC mRNA was determined by real-time PCR.

FIG. 3E depicts the effects of CUDC-907 on ACTH secretion in corticotroph tumor primary cultures. The effects of CUDC-907 on ACTH secretion was determined by real-time ELISA.

FIG. 4A depicts the effects of CUDC-907 in a xenograft model of Cushing disease; AtT20 cells inoculated into athymic nude mice (Nu/J strain, Jackson lab) and CUDC-907 (300 mg/kg dissolved in Captisol) was administered daily by oral gavage for 18 days. Animal bodyweight was recorded daily.

FIG. 4B depicts the effects of CUDC-907 in a xenograft model of Cushing disease; AtT20 cells inoculated into athymic nude mice (Nu/J strain, Jackson lab) and CUDC-907 (300 mg/kg dissolved in Captisol) was administered daily by oral gavage for 18 days. Tumor size was recorded daily.

FIG. 4C depicts the effects of CUDC-907 in a xenograft model of Cushing disease; AtT20 cells inoculated into athymic nude mice (Nu/J strain, Jackson lab) and CUDC-907 (300 mg/kg dissolved in Captisol) was administered daily by oral gavage for 18 days. Tumor size was measured post euthanizer.

FIG. 4D depicts the effects of CUDC-907 in a xenograft model of Cushing disease; AtT20 cells inoculated into athymic nude mice (Nu/J strain, Jackson lab) and CUDC-907 (300 mg/kg dissolved in Captisol) was administered daily by oral gavage for 18 days. Tumor weight was measured post euthanizer.

FIG. 4E depicts the effects of CUDC-907 in a xenograft model of Cushing disease; AtT20 cells inoculated into athymic nude mice (Nu/J strain, Jackson lab) and CUDC-907 (300 mg/kg dissolved in Captisol) was administered daily by oral gavage for 18 days. Blood samples were collected by cardiac puncture and plasma ACTH levels were measured by ELISA.

FIG. 4F depicts the effects of CUDC-907 in a xenograft model of Cushing disease; AtT20 cells inoculated into athymic nude mice (Nu/J strain, Jackson lab) and CUDC-907 (300 mg/kg dissolved in Captisol) was administered daily by oral gavage for 18 days. Blood samples were collected by cardiac puncture and plasma corticosterone levels were measured by ELISA.

FIG. 5A depicts the development of A Highly Sensitive Assay to Identify Inhibitors of Corticotroph Tumor ACTH Secretion. Schematic overview of the novel ACTH AlphaLISA assay. Streptavidin-labelled donor beads and anti-mouse IgG coated acceptor beads are brought into close proximity by biotinylated ACTH peptide and mouse anti-ACTH antibody. Laser excitation triggers transfer of singlet oxygen from donor to acceptor beads producing a signal (Upper). Presence of ACTH analyste in cell supernatant displaces the donor and acceptor beads to inhibit the Alpha signal (Lower).

FIG. 5B depicts the development of A Highly Sensitive Assay to Identify Inhibitors of Corticotroph Tumor ACTH Secretion. Anti-ACTH antibody configuration using three anti-ACTH antibodies (1 nM, #1 EMD Cat. CBL57; #2 Abcam Cat. Ab20358; #3 Novus Cat. NBP2-34529) in combination with biotinylated ACTH peptide (1-20 nM). Antibody #1 ( [symbol] ) generated robust AlphaLISA signal at low biotinylated ACTH concentrations, and was selected for further assay development.

FIG. 5C depicts the development of A Highly Sensitive Assay to Identify Inhibitors of Coticotroph Tumor ACTH Secretion. To optimize cell supernatant (SN) volume and anti-ACTH antibody and peptide concentrations, varying volumes of 3-day (3D) and 4-day (4 D) AtT20 cell SNs in combination with biotinylated ACTH peptide (Biotin-ACTH Peptide, 0.1 & 0.3 nM) and anti-ACTH antibody (αACTH Ab, 0.1 & 0.3 nM) were compared. Based on raw AlphaLISA signals (Upper) and Z′ values (Lower), a stable assay performance was observed with a 3D culture time in combination with 0.3 nM Biotin-ACTH Peptide and 0.1 nM aACTH Ab across a 8-fold SN volume range (2.5-20 mL, boxed).

FIG. 5D depicts the development of A Highly Sensitive Assay to Identify Inhibitors of Corticotroph Tumor ACTH Secretion. The assay volumes tested (20, 15, 10 and 5 mL) demonstrated potent inhibition of AlphaLISA signals generating a Z′ factor >0.6, so a 5 mL assay volume was selected (boxed).

FIG. 5E depicts the development of A Highly Sensitive Assay to Identify Inhibitors of Corticotroph Tumor ACTH Secretion. Using the 5 mL reaction volume with 2 mL of 3D AtT20 cell SN, basal Alpha signal reduced in a stepwise fashion with decreasing acceptor bead concentrations (8-4 mg/mL), but Z′ remained >0.7 for all, so 4 mg/mL acceptor bead was selected (Left Panel, boxed). Using this acceptor bead concentration, the Z′ factor dropped below 0.7 when the donor bead concentration was reduced from 10 to 5 mg/mL, so 10 mg/mL donor bead was chosen (Right Panel, boxed).

FIG. 5F depicts the development of A Highly Sensitive Assay to Identify Inhibitors of Corticotroph Tumor ACTH Secretion. Commercial ImmunoAssay Buffer supplemented with 0.1, 0.5 and 1% BSA demonstrated best signal stability with 0.5% BSA as it generated a Z′ factor constantly >0.7 (θ), so 0.5% BSA was selected as buffer supplement.

FIG. 5G depicts the development of a sensitive assay to identify inhibitors of corticotroph tumor acth secretion. Summary of optimal gnal assay component volumes, concentrations and procedures for the ACTH AlphaLiSA.

FIG. 6A depicts the chemical structure of CUDC-907 with the anti-HDAC hydroxamate moiety and the PI3K inhibitor skeleton.

FIG. 6B depicts the results of a study wherein AtT20 cells were treated with CUDC-907 or the reference compounds (panobinostat, vorinostat, buparlisib, and pictilisib) at a concentration of 40 nM to 380 μM. The EC₅₀ for ACTH secretion inhibition were calculated using a sigmoidal dose-response curve (GraphPad Prism).

FIG. 6C depicts the results of a study wherein AtT20 cells were treated with CUDC-907 or the reference compounds at a concentration of 10 μM to 380 μM for 3 days, after which IC₅₀ for proliferation inhibition was calculated.

FIG. 6D depicts the results of a study wherein AtT20 cells were stably transfected with a POMC promoter driven luciferase (POMC-Luc) and then treated with CUDC-907 or the reference compounds (10 μM to 380 μM, 2-fold dilution) for 1 day, after which the EC₅₀ for POMC transcription inhibition was calculated.

FIG. 6E depicts the results of a study wherein AtT20 cells were treated with CUDC-907 or the reference compounds at a range of concentrations from 20 nM to 1.25 nM (2-fold dilution) for 24 h and the POMC mRNA expression were detected by real time PCR.

FIG. 6F depicts the results of a study wherein AtT20 cells were treated with panobinostat and burparlisib simultaneously to detect the effects on ACTH secretion.

FIG. 6G depicts the results of a study wherein AtT20 cells were treated with panobinostat and burparlisib simultaneously to detect the effects on cell proliferation.

FIG. 6H depicts the results of a study wherein AtT20 cells were treated with panobinostat and burparlisib simultaneously to detect the effects on POMC transcription.

FIG. 6I depicts the results of a study wherein AtT20 cells were treated with panobinostat and burparlisib simultaneously to detect the effects on POMC mRNA expression.

FIG. 7A depicts the change in expression expression of known positive and negative POMC regulators following CUDC-907 treatment.

FIG. 7B depicts the results of a study wherein AtT20 cells were transiently transfected with Nur factors to determine the factors involved in CUDC-907 actions.

FIG. 7C depicts the results of a study wherein AtT20 cells were transiently transfected with LXRs to determine the factors involved in CUDC-907 actions.

FIG. 7D depicts the acute effect of CUDC-907 (6 h) on expression of Nurr1.

FIG. 7E depicts mRNA expression of Nurr1, as measured by real time PCR, following CUDC-907 treatment as indicated.

FIG. 7F depicts the interactions between Nurr1, HDACs, and Nurr1 phosphorylation, as detected by immunoprecipitation.

FIG. 8A depicts a study wherein AtT20 cells were treated with 5 nM CUDC-907 for 24 h after which c-Myc and cell cycle inhibitor mRNA levels were quantified (CDKN1A, 1B, and 1C) by real time PCR.

FIG. 8B depicts histone acetylation (H3K9) and p27 expression in AtT20 cells following treatment with CUDC-907 for 3 days.

FIG. 8C depicts histone acetylation (H3K9) and AKT pathway activation in AtT20 cells following treatment with CUDC-907 for 3 days.

FIG. 8D depicts murine corticotroph tumor Caspase-3/7 activation, as determined by an Caspase-Glo3/7 assay (Promega), following incubation with CUDC-907 for 24 hours.

### DETAILED DESCRIPTION OF THE INVENTION

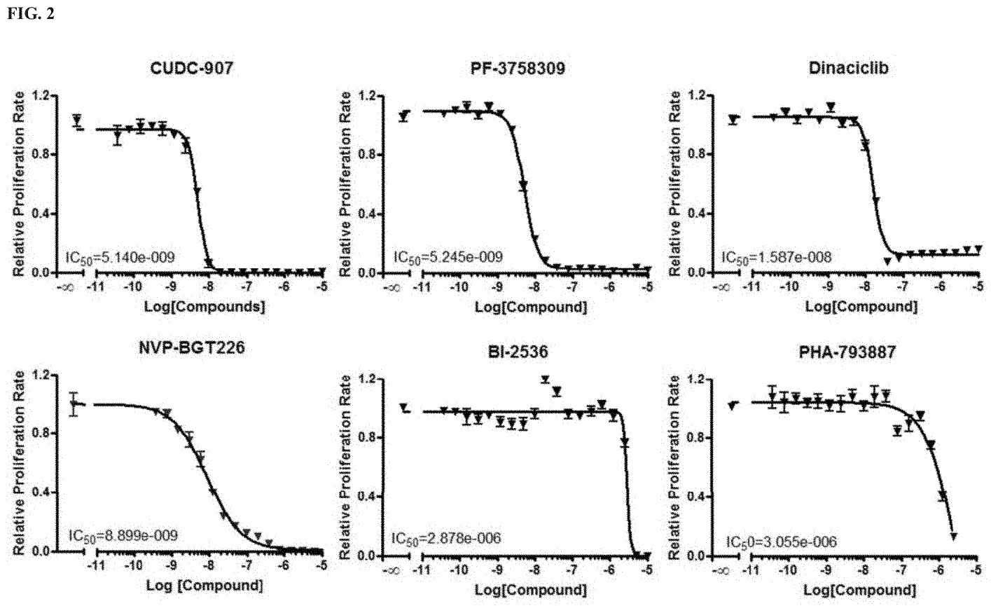

In one aspect, the invention provides a “gain of signal” ACTH AlphaLISA assay useful for a high throughput screen (HTS) evaluation. Using ACTH AlphaLISA assay described herein in combination with nuclei staining (Hoechst 33342 dye), the inventors screened an annotated kinase inhibitor library. The inventors determined the IC₅₀ of the most potent 6 compounds using CellTiter-Glo and found that CUDC-907 exhibited the greatest anti-proliferation effects with IC₅₀ of 5.1 nM at 3 day-treatment (FIG. 2).

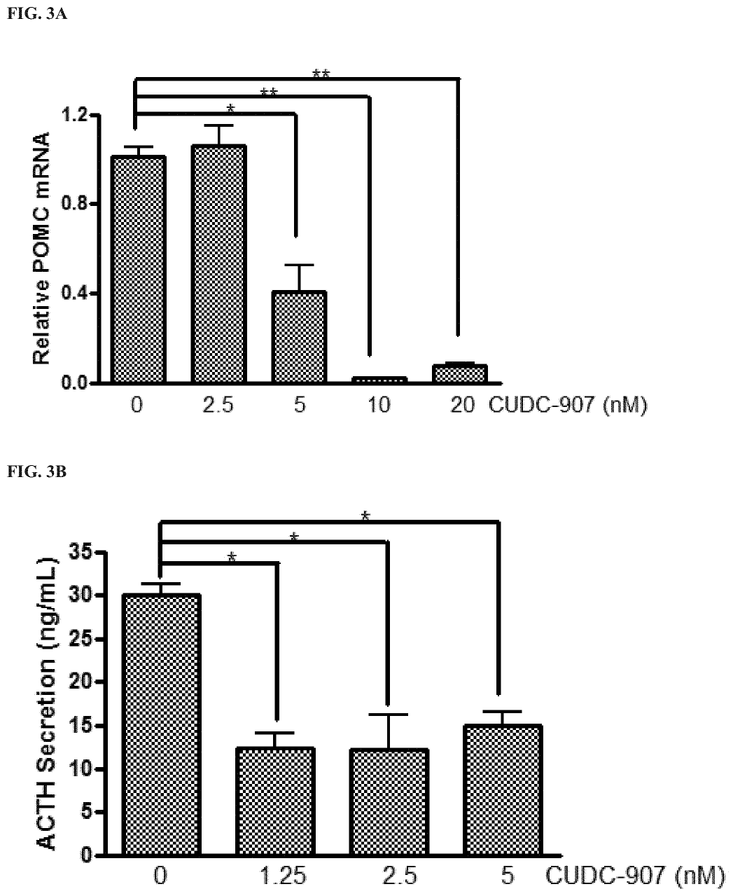

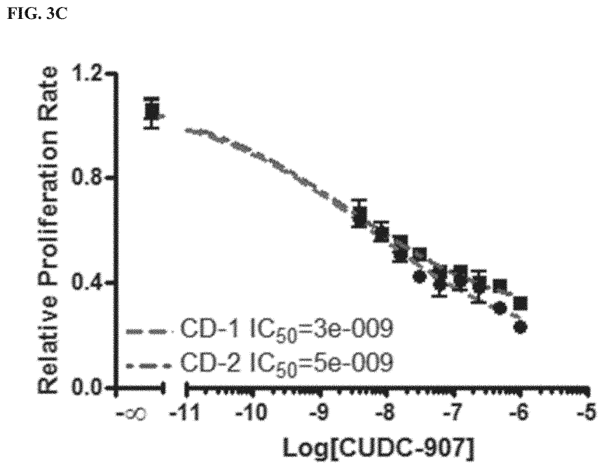

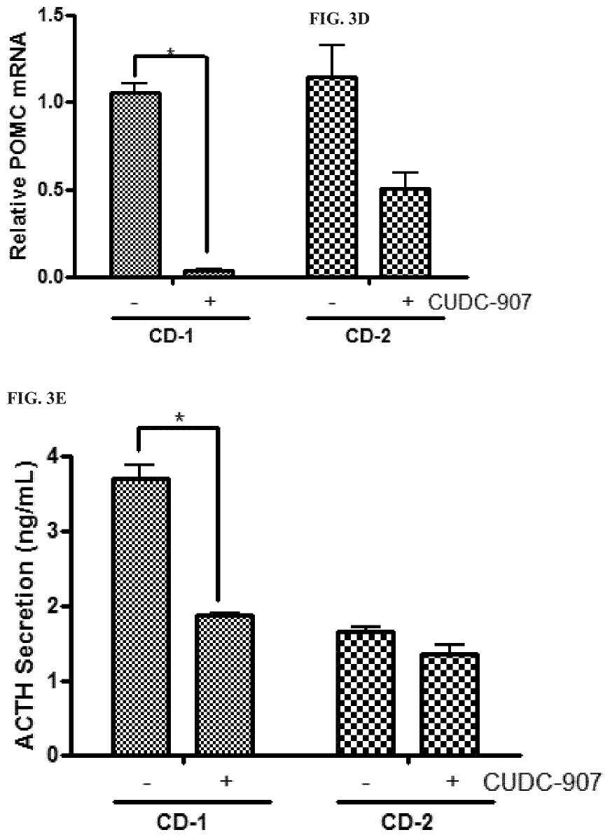

CUDC-907 is a novel oral dual inhibitor of class 1 phosphoinositide 3-kinase (PI3K; α, β, and δ isoforms) as well as histone deacetylase (HDAC; class I and II) enzymes, with an IC₅₀ of 19/54/39 nM and 1.7/5.0/1.8/2.8 nM for PI3Kα/PI3Kβ/PI3Kδ and HDAC1/HDAC2/HDAC3/HDAC10, respectively (Qian et al. 2012). To determine the mode of actions of CUDC-907 in inhibition of ACTH secretion in AtT20 cells, the changes of POMC mRNA expression and ACTH secretion following CUDC-907 treatment were evaluated. The real-time PCR results indicated that CUDC-907 treatment resulted in reduction of POMC mRNA at doses of 5 nM and higher (Relative POMC mRNA, Vehicle 1.0±0.04, CUDC-907 5 nM 0.4±0.12, p<0.05; CUDC-907 10 nM 0.02±0.001, p<0.01; CUDC-907 20 nM 0.08±0.009, p<0.01, FIG. 3A). The inhibitory effect of CUDC-907 on ACTH secretion was more dramatic compared to its effect on POMC mRNA with 59% reduction at 1.25 nM (ACTH secretion (ng/mL), Vehicle 30±19, CUDC-907 1.25 nM 12±2.4, p<0.05; CUDC-907 2.5 nM 12±5.7, p<0.05; CUDC-907 15±2.2, p<0.05, FIG. 3B), indicating that CUDC-907 at lower doses inhibits ACTH secretion independent on its effect on POMC mRNA synthesis and cell proliferation blockage.

The effects of CUDC-907 in human corticotroph tumor primary cultures (n=2) were also measured. As shown in FIG. 3C, CUDC-907 exhibited comparable anti-proliferative effect with IC₅₀ of 3 nM and 5 nM in two human primary corticotroph tumor cultures. Additionally, CUDC-907 inhibited POMC mRNA expression (Relative POMC mRNA, Vehicle 1.0±0.06, CUDC-907 0.03±0.01, p<0.05, FIG. 3D) and ACTH secretion (ACTH secretion (ng/mL), Vehicle 3.7±0.2, CUDC-907 1.9±0.001, p<0.05, FIG. 3E) respectively in a human corticotroph primary culture.

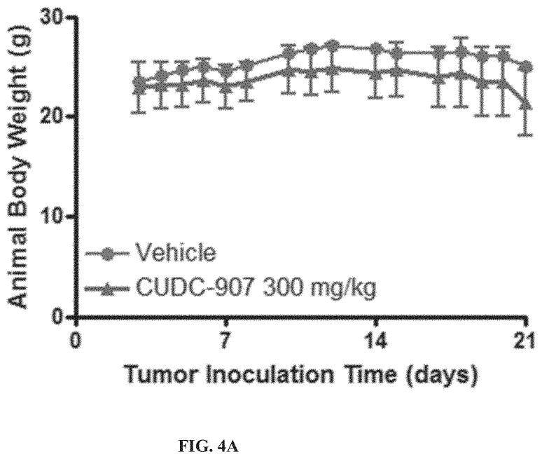

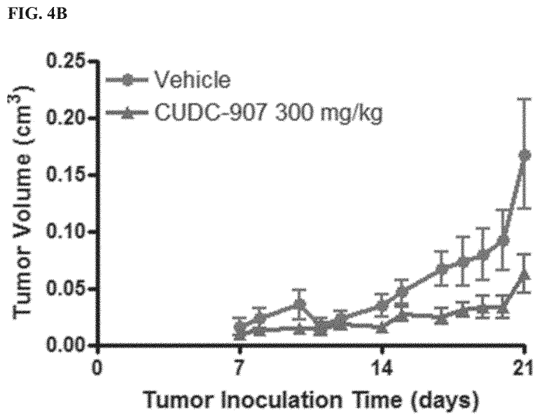

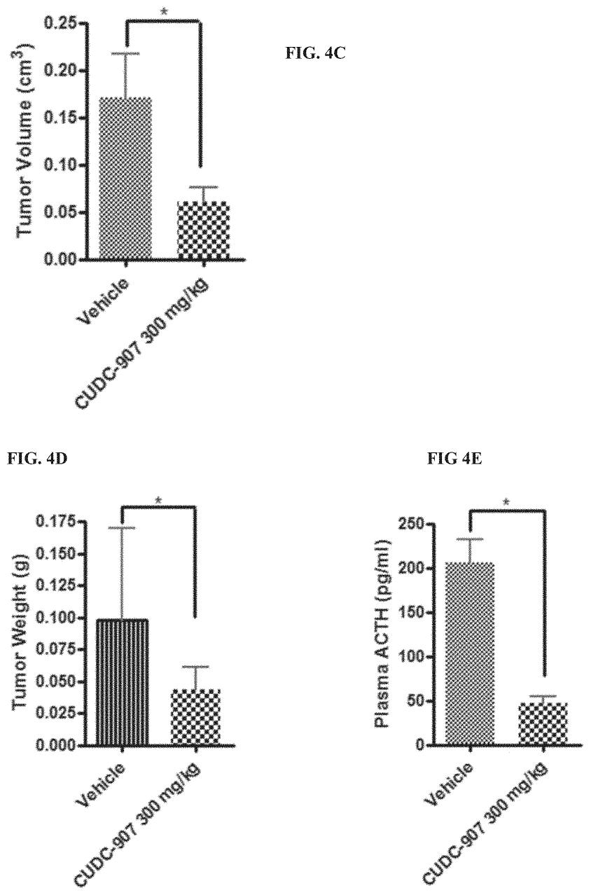

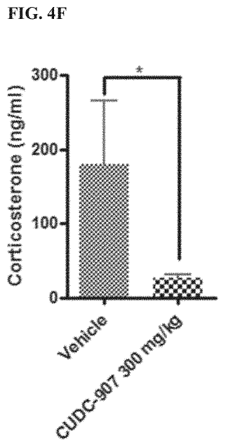

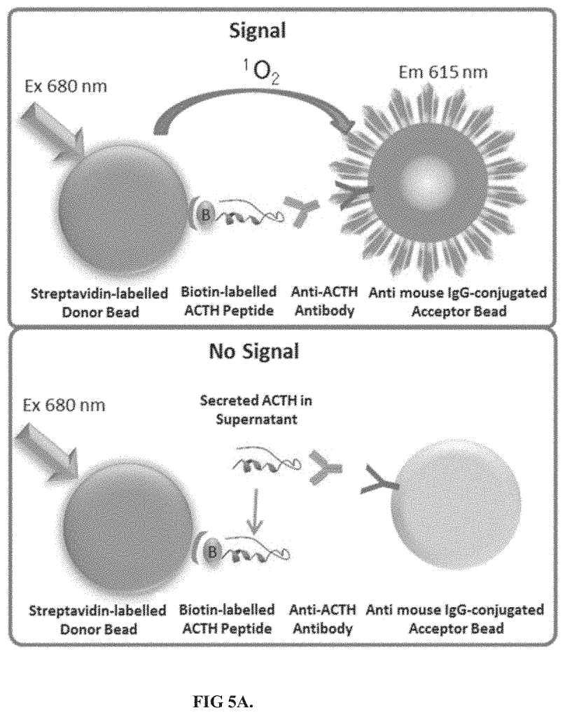

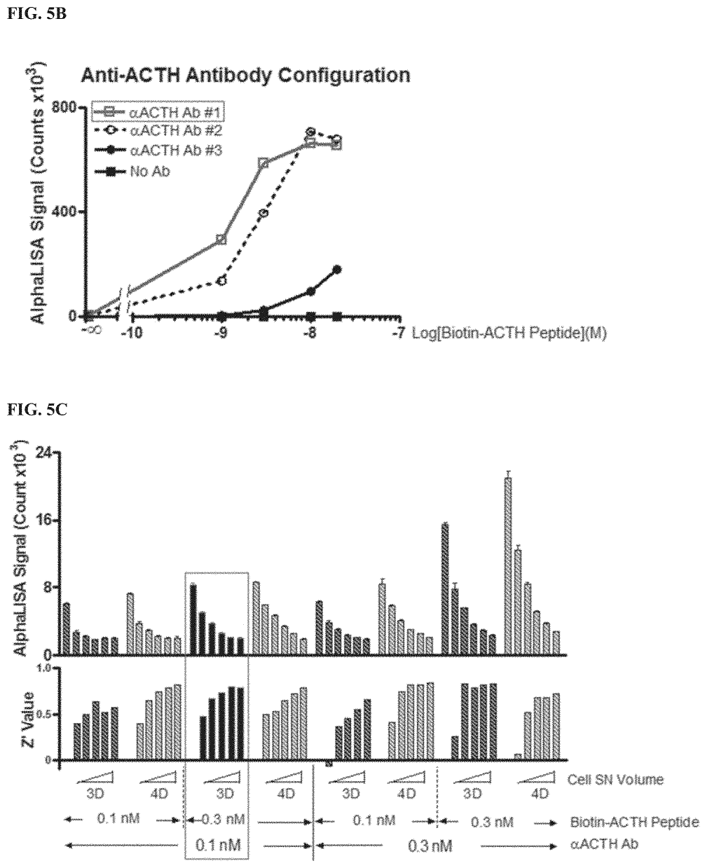

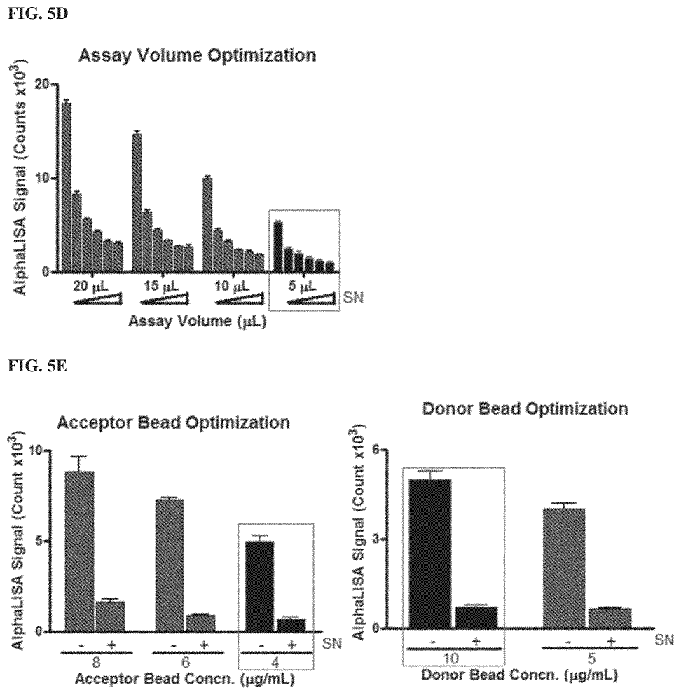

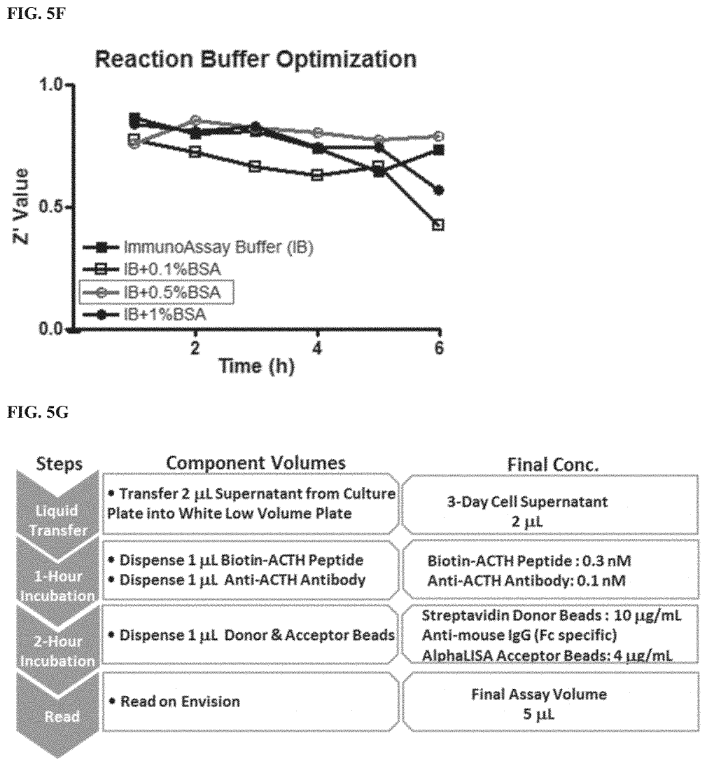

The in vivo anti-tumor effect of CUDC-907 was also evaluated using a CD xenograft model. AtT20 cells (2×10⁵/animal) were injected into the hind flank region of athymic nude mice (Nu/J strain from Jackson lab). Three days post inoculation, animals were randomly assigned to receive either vehicle (n=10) or CUDC-907 (n=10). CUDC-907 was dissolved in Captisol (Cydex Pharmaceuticals) by vortex and sonication and administered via oral gavage at the dose of 300 mg/kg. During 18-days of CUDC-907-treatment, animal bodyweight (FIG. 4A) and tumor sizes (FIG. 4B) were measured daily. Upon experiment completion, the animals were euthanized and tumors were harvested, and weighed. Blood samples were collected by cardiac puncture. CUDC-907 treatment resulted in a ˜35% reduction in tumor size and 44% reduction in tumor weight (tumor volume (cm³), Control 0.17±0.05 vs. CUDC-907 0.065±0.02, p<0.05, FIG. 4C) and 44% (tumor weight (gram), Control 0.098±0.02 vs. CUDC-907 0.04±0.006, p<0.05, FIG. 4D) compared to vehicle controls. Plasma ACTH (ACTH (pg/mL) Control 206.1±27.2 vs. CUDC-907 47.4±7.3, p<0.05, FIG. 4E) and corticosterone (Corticosterone (ng/mL) Control 180±87 vs. CUDC-907 27±4.66, p<0.05, FIG. 4F) levels were reduced by 77% and 85% respectively in CUDC-907 treated mice compared to controls.

Given the surprisingly efficacy demonstrated in in vitro and in vivo models of CD, CUDC-907 and related compounds can be used to treat CD.

In certain aspects, the present disclosure provides a method of treating a disease or disorder characterized by an increased secretion of adrenocorticotropic hormone, comprising administering, to a subject in need thereof, a compound that inhibits both the secretion of adrenocorticotropic hormone and tumor growth. In certain embodiments, the disease or disorder is hypercortisolism, Itsenko-Cushing syndrome, hyperadrenocorticism, or Cushing's Syndrome. In certain preferred embodiments, the disease or disorder is Cushing's disease.

In another aspect, the present disclosure provides a method of treating Cushing's disease, comprising administering, to a subject in need thereof, a compound that inhibits both the secretion of adrenocorticotropic hormone and tumor growth.

In certain embodiments, the compound is a PI3K inhibitor, a HDAC inhibitor, a PKA inhibitor, a CDK inhibitor, a AKT inhibitor, a mTOR inhibitor, a PLK inhibitor, a cell cycle inhibitor, or an inhibitor of cytoskeletal signaling. In certain embodiments, the compound is a PI3K inhibitor, a HDAC inhibitor, a PKA inhibitor, a CDK inhibitor, a AKT inhibitor, a mTOR inhibitor, or a PLK inhibitor. In certain embodiments, the compound is a PI3K inhibitor. In certain embodiments, the compound is a PKA inhibitor. In certain embodiments, the compound is a CDK inhibitor. In certain embodiments, the compound is an AKT inhibitor. In certain embodiments, the compound is a mTOR inhibitor. In certain embodiments, the compound is a PLK inhibitor. In certain preferred embodiments, the compound is a PI3K inhibitor. In certain preferred embodiments, the compound is an HDAC inhibitor. In certain particularly preferred embodiments, the compound is a both a PI3K inhibitor and an HDAC inhibitor.

In certain embodiments, the compound is CUDC-907

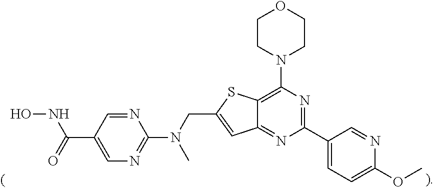

In certain embodiments, the compound is a pharmaceutical salt of CUDC-907. CUDC-907 and related compounds and methods are described in U.S. Pat. No. 8,710,219, the contents of which are fully incorporated by reference herein.

In certain embodiments, the compound is PF-3758309

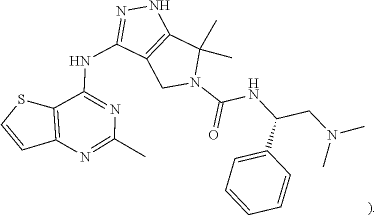 [SMILES: Cc1nc(Nc2n[nH]c3c2CN(C(=O)N[C@H](CN(C)C)c2ccccc2)C3(C)C)c2sccc2n1]

In certain embodiments, the compound is a pharmaceutical salt of PF-3758309. PF-3758309 and related compounds and methods are described in U.S. Pat. No. 8,067,591, the contents of which are fully incorporated by reference herein.

In certain embodiments, the compound is Dinaciclib

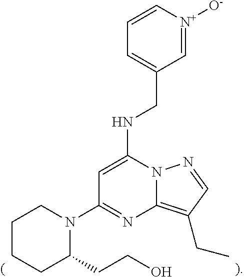 [SMILES: CCc1cnn2c(NCc3ccc[n+]([O-])c3)cc(N3CCCC[C@H]3CCO)nc12]

In certain embodiments, the compound is a pharmaceutical salt of Dinaciclib. Dinaciclib and related compounds and methods are described in U.S. Pat. No. 8,076,479, the contents of which are fully incorporated by reference herein.

In certain embodiments, the compound is BGT226

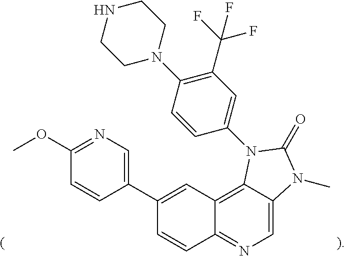

In certain embodiments, the compound is a pharmaceutical salt of BGT226. BGT226 and related compounds and methods are described in U.S. Pat. No. 8,034,816, the contents of which are fully incorporated by reference herein.

In certain embodiments, the compound is BI 2536

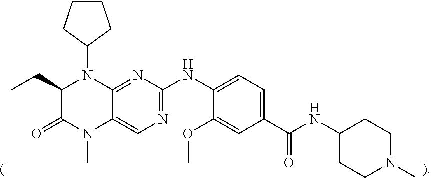 [SMILES: CC[C@@H]1C(=O)N(C)c2cnc(Nc3ccc(C(=O)NC4CCN(C)CC4)cc3OC)nc2N1C1CCCC1]

In certain embodiments, the compound is a pharmaceutical salt of BI 2536. BI 2536 and related compounds and methods are described in U.S. Pat. No. 7,667,039, the contents of which are fully incorporated by reference herein.

In certain embodiments, the compound is PHA-793887

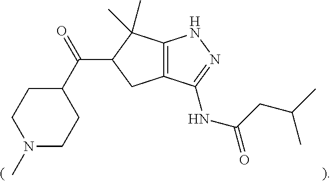 [SMILES: CC(C)CC(=O)Nc1n[nH]c2c1CC(C(=O)C1CCN(C)CC1)C2(C)C]

In certain embodiments, the compound is a pharmaceutical salt of PHA-793887. PHA-793887 and related compounds and methods are described in U.S. Pat. No. 7,407,971, the contents of which are fully incorporated by reference herein.

In certain embodiments, the method further comprises administering at least one additional compound. In certain embodiments, the method further comprises administering at least two additional compounds. In certain embodiments, the method further comprises administering one additional compound. In certain embodiments, the method further comprises administering two additional compounds. In certain embodiments, the additional compound is a PI3K inhibitor, a HDAC inhibitor, a PKA inhibitor, a CDK inhibitor, a AKT inhibitor, a mTOR inhibitor, a PLK inhibitor, a cell cycle inhibitor, or an inhibitor of cytoskeletal signaling. In certain preferred embodiments, the method comprises administering a combination of a PI3K inhibitor and a HDAC inhibitor. In certain embodiments, the additional compound is

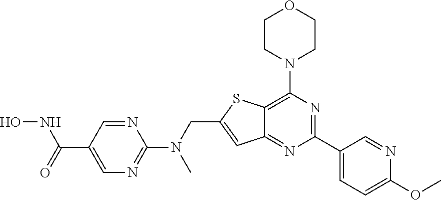 [SMILES: COc1ccc(-c2nc(N3CCOCC3)c3sc(CN(C)c4ncc(C(=O)NO)cn4)cc3n2)cn1]

or a pharmaceutically acceptable salt thereof. In certain embodiments, the additional compound is

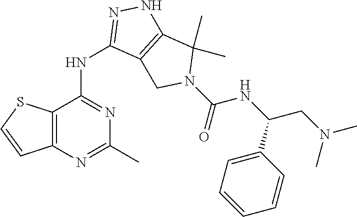 [SMILES: Cc1nc(Nc2n[nH]c3c2CN(C(=O)N[C@H](CN(C)C)c2ccccc2)C3(C)C)c2sccc2n1]

or a pharmaceutically acceptable salt thereof. In certain embodiments, the additional compound is

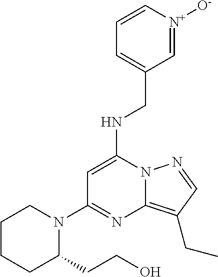 [SMILES: CCc1cnn2c(NCc3ccc[n+]([O-])c3)cc(N3CCCC[C@H]3CCO)nc12]

or a pharmaceutically acceptable salt thereof. In certain embodiments, the additional compound is

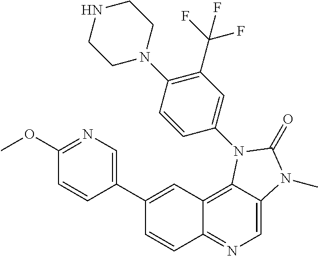

or a pharmaceutically acceptable salt thereof. In certain embodiments, the additional compound is

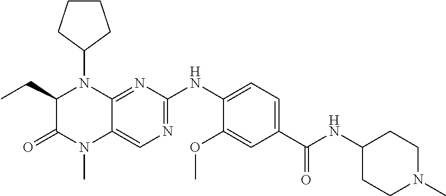 [SMILES: CC[C@@H]1C(=O)N(C)c2cnc(Nc3ccc(C(=O)NC4CCN(C)CC4)cc3OC)nc2N1C1CCCC1]

or a pharmaceutically acceptable salt thereof. In certain embodiments, the additional compound is

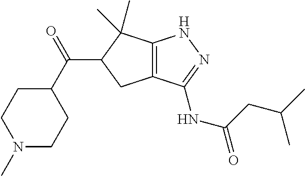 [SMILES: CC(C)CC(=O)Nc1n[nH]c2c1CC(C(=O)C1CCN(C)CC1)C2(C)C]

or a pharmaceutically acceptable salt thereof.

In certain embodiments, the method is performed continuously for at least 12 months. In certain embodiments, the method is performed continuously for at least 24 months.

In certain aspects, the present disclosure provides a method of identifying a compound that inhibits the secretion of adrenocorticotropic hormone (ACTH) and tumor growth, comprising the steps of:
a) contacting a plurality of AtT20 cells with the compound, thereby forming an assay mixture;
b) quantifying the ability of the compound to inhibit the secretion of ACTH; and
c) quantifying the ability of the compound to inhibit nuclei;
wherein the compound is identified as being an inhibitor of ACTH secretion and tumor growth if the compound inhibits both ACTH secretion and nuclei growth at a predetermined threshold.

In certain embodiments, step b) is performed about 1 day, about 2 days, about 3 days, about 4 days, about 5 days, about 6 days, or about 7 days after step a).

In certain embodiments, quantifying the ability of the compound to inhibit the secretion of ACTH comprises the steps of:
i) transferring a first volume of the assay mixture to a sample well;
ii) contacting the sample well with an ACTH peptide solution and an anti-ACTH antibody solution, thereby forming an analysis mixture;
iii) contacting the analysis mixture with a plurality of donor beads and a plurality of acceptor beads;
iv) transferring the analysis mixture to an AlphaLISA signal detector; and
v) detecting the AlphaLISA signal.

In certain embodiments, the ACTH peptide is a biotin labeled ACTH peptide. In certain preferred embodiments, the ACTH peptide is a biotin labeled human (1-39aa) ACTH peptide.

In certain embodiments, the anti-ACTH antibody is a monoclonal anti-ACTH antibody. In certain embodiments, the anti-ACTH antibody is an anti-ACTH (1-24aa) monoclonal antibody. In certain embodiments, the antibody is a mouse anti-ACTH (1-24aa) monoclonal antibody.

In certain embodiments, the donor beads are labelled with streptavidin. In certain embodiments, the donor beads bind to the biotin-labelled ACTH peptide. In certain embodiments, the donor beads are labelled with anti-mouse IgG.

In certain embodiments, detecting the AlphaLISA signal comprises contacting the analysis mixture with red light. In certain embodiments, the red light has a wavelength of about 680 nm.

In certain embodiments, the concentration of the ACTH peptide is about 0.1 nM, about 0.2 nM, about 0.3 nM, about 0.4 nM, or about 0.5 nM. In certain embodiments, the concentration of the ACTH peptide is about 0.3 nM.

In certain embodiments, the concentration of the antibody is about 0.1 nM, about 0.2 nM, about 0.3 nM, about 0.4 nM, or about 0.5 nM. In certain embodiments, the concentration of the antibody is about 0.1 nM.

In certain embodiments, the concentration of the acceptor beads is about 10 μg/mL, about 8 μg/mL, about 6 μg/mL, about 4 μg/mL, or about 2 μg/mL. In certain embodiments, the concentration of the acceptor beads is about 4 μg/mL. In certain embodiments, the concentration of the acceptor beads is about 25 μg/mL, about 20 μg/mL, about 16 μg/mL, about 10 μg/mL, or about 5 μg/mL.

In certain embodiments, quantifying the ability of the compound to inhibit the secretion of ACTH comprises the steps of:

i′) contacting the assay mixture with a dye;

ii′) imaging the assay mixture; and

iii′) analyzing the image.

In certain embodiments, the dye is Hoechst 33342.

In certain embodiments, the concentration of the dye is about 10 μg/mL, about 8 μg/mL, about 6 μg/mL, about 4 μg/mL, or about 2 μg/mL. In certain embodiments, the concentration of the dye is about 2 μg/mL.

In certain embodiments, after step i′) but before step ii′) the assay mixture is incubated.

In certain embodiments, the Z′ factor of the method is above 0.5, above 0.6, above 0.7, above 0.8, or above 0.9. In certain embodiments, the Z′ factor of the method is above 0.8. In certain embodiments, the Z′ factor of the method is about 0.88.

In certain embodiments, the predetermined threshold is an ACTH inhibition percentage of at least 50%, at least 60%, at least 70%, or at least 80% and a nuclei inhibition percentage of at least 70%, at least 80%, at least 85%, at least 90%, at least 95%, at least 98% or at least 99%. In certain embodiments, the predetermined threshold is an ACTH inhibition percentage of at least 80% and a nuclei inhibition percentage of at least 85%, at least 90%, at least 95%, at least 98%, or at least 99%. In certain embodiments, the predetermined threshold is an ACTH inhibition percentage of at least 80% and a nuclei inhibition percentage of at least 95%, at least 98%, or at least 99%. In certain embodiments, the predetermined threshold is an ACTH inhibition percentage of at least 80% and a nuclei inhibition percentage of at least 98% or at least 99%.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self emulsifying drug delivery system or a self microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals.

A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required.

For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject.

Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, I-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, l-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, Calif. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(OXO-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

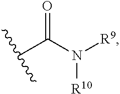

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

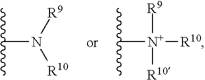

wherein R⁹, R¹⁰, and R¹⁰′ each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

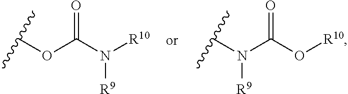

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical.

Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

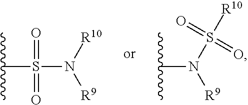

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

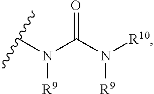

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

### EXAMPLES

The invention now being generally described, it will be more readily understood by reference to the following examples which are included merely for purposes of illustration of certain aspects and embodiments of the present invention, and are not intended to limit the invention.

### Example 1: Development of an Assay to Identify Dual Inhibitors of Corticotroph Tumor Growth and ACTH Secretion

ACTH is a highly conserved 39aa peptide (human and mouse ACTH differ by only 2 amino acids) which is synthesized primarily in anterior pituitary corticotroph cells. Under physiological conditions, circulating ACTH binds its receptor on the adrenal cortex to regulate glucocorticoid synthesis and secretion. Commercial ACTH immunoassays are typically 96-well format Sandwich ELISAs, require a large sample volume (200 μL), a handling time >4½ hours and cost ˜$5 per reaction. This format is not ideal for automated large scale screening so the inventors developed a novel “gain of signal” homogenous ACTH AlphaLISA assay.

In the ACTH AlphaLISA assay described herein, streptavidin-labelled donor beads bind strongly to biotin-labelled ACTH peptide (human, 1-39aa), which is then captured by a mouse anti-ACTH (1-24aa) monoclonal antibody. The latter mouse antibody is then trapped by an anti-mouse IgG (Fc specific) conjugated to acceptor beads, bringing the donor and acceptor beads into close proximity. Upon laser excitation of the donor beads at 680 nm, a short-lived singlet oxygen molecule is produced and interacts with adjacent acceptor beads to generate an amplified chemiluminescent signal at 615 nm (summarized schematically in FIG. 3A, Upper Panel). If a cell culture supernatant (SN) containing secreted ACTH is added to the assay, the ACTH competes with the biotinylated ACTH peptide to bind the anti-ACTH antibody thereby disrupting proximity of the donor and acceptor to inhibit signal emission (FIG. 3A, Lower Panel). Conversely if the cell SN ACTH is reduced following treatment with an ACTH inhibitor, the Alpha signal is restored.

### ACTH AlphaLISA Assay Development and Optimization

Monoclonal anti-ACTH antibodies were exclusively generated against the N-terminus 1-24 aa ACTH sequences. The inventors compared 3 individual anti-ACTH antibody (1 nM) with increasing concentrations of biotinylated ACTH peptide (AnaSpec) that brought the streptavidin-labelled donor beads and the anti-mouse IgG (Fc specific)-labeled acceptor beads (PerkinElmer) into close proximity to generate a dose-dependent increased Alpha signal (FIG. 3B). Antibody #1 (EMD, Cat. CBL57) exhibited a robust Alpha signal (even at low biotinylated-ACTH concentrations) and thus was chosen for further assay development (FIG. 3B).

To determine the optimal assay conditions, the inventors tested biotinylated-ACTH peptide at concentrations of 0.1 & 0.3 nM in combination with anti-ACTH antibody of 0.1 & 0.3 nM with varying volumes of 3- and 4-day (D) murine corticotroph tumor cell derived SNs (ACTH Concn. ˜10⁻¹⁰M). AlphaLISA signals displayed that both the 3- and 4-D SN exhibited a robust dose- (SN volume-) dependent reduction in the competition assay (FIG. 3C). The inventors then calculated the assay Z′ factor, a statistical parameter calculated from the standard deviations of negative and positive controls to assess assay performance and facilitate assay optimization. The Z′ factor remained consistently >0.7 using 0.3 nM biotinylated-ACTH peptide (Biotin-ACTH Peptide) in combination with 0.1 nM anti-ACTH antibody (aACTH-Ab) with the 3- and 4-D SN (except the lowest volume)(FIG. 3C). Due to potential compound instability with longer incubation periods, 0.3 nM of biotinylated ACTH peptide, 0.1 nM of anti-ACTH Ab and a 3-D corticotroph tumor SN were selected as optimal assay conditions (FIG. 3C).

The inventors then tested 20, 15, 10, and 5 μL volumes to find the lowest effective assay format to optimize cost. 3-day AtT20 corticotroph tumor cell SN generated potent inhibition of Alpha signals maintaining a Z′ factor >0.6 for all assay volumes tested and accordingly, 5 μL was chosen for further validation (FIG. 3D). Several acceptor bead concentrations (8, 6 and 4 μg/mL) were evaluated. The basal Alpha signal proportionally reduced with lower acceptor bead concentrations, but the Z′ factor still remained >0.7 for all and 4 μg/mL acceptor bead concentration was selected (FIG. 3E). When the donor bead concentration was reduced from 10 to 5 μg/mL, Z′ factor dropped from 0.88 to 0.65. Accordingly, a donor bead concentration of 10 μg/mL was chosen (FIG. 3E). Stability of the aACTH-Ab/Biotin-ACTH-peptide interaction was tested using commercial immune-assay buffer (IB, Perkin Elmer) in combination with various BSA concentrations (0.1-1%) and the inventors have demonstrated that the presence of 0.5% BSA provided optimal buffer conditions (FIG. 3F). Employing the validated assay conditions (in FIG. 3G), the final assay comprises: a liquid transfer step of 2 μL of supernatant, followed by addition of 1 μL of biotinylated-ACTH peptide and 1 μL of anti-ACTH antibody with 1 h incubation, followed by addition of 1 μL of donor and acceptor bead mixture solution with a 2 h incubation for a total of 3 h assay time. An optical plate seal to minimize potential evaporation further enhanced the Z′ factor during assay incubation (not shown). The approximate cost of the AlphaLISA assay described herein is ˜$0.1 per reaction; notably, this is 50 times less than commercial ACTH ELISAs.

### Assay Protocol

AtT20 cells were plated on 384 well black plates (columns 1 to 22) at a density of 1,500 cells/well using a Multidrop 384 (Thermo). One column (#1) with vehicle treatment only, and two cell-free columns (#23 & 24) were included as AlphaLISA signal negative and positive controls respectively to monitor assay performance. Dexamethasone, a synthetic glucocorticoid that potently inhibits ACTH secretion was added to column 2 (#2) as a reference compound to monitor cell response and reassure assay performance. The test compounds were added using a Biomek FX (Beckman Coulter) with a 384 custom pin tool (V&P Scientific) into columns 3 to 22, following which the cells and compounds are incubated in a Cytomat 6000, sealed with a gas permeable polyurethane film (USA Scientific) for 3 days.

Thereafter, 2 μL of cell culture supernatant was transferred from the black culture plate into a white low volume assay plate (Corning) using Vprep (Agilent) equipped with Velocity 11 Biocel 1800 (Agilent). 1 μL of biotinylated ACTH peptide solution (AnaSpec) and 1 μL of anti-ACTH antibody solution (EMD) is added sequentially into the low volume assay plate using AquaMax DW4 (Molecular Devices) Following a 1 h incubation, 1 μL donor and acceptor beads (PerkinElmer) were added to the assay plate using ELx405 (BioTek). Optical seals (Sigma) are used to minimize assay evaporation during the subsequent 2 h incubation. The assay plate is then transferred by a Thermo Spinnaker robotic arm to Envision (PerkinElmer) for AlphaLISA signal detection.

In parallel, Hoechst 33342 dye (Invitrogen) is dispensed onto the source plate using a Multidrop 384 (Thermo) to reach a working concentration of 2 μg/mL for nuclei staining. This plate was incubated in a Cytomat 6000 sealed with gas permeable polyurethane film (USA Scientific) and imaged on ImageXpress^{XL} (Molecular Devices). All these instruments are integrated on a Beckman Coulter SAMI automation platform which tracks timing to ensure accuracy and consistency of all steps.

### Example 2: Screening of Exemplary Compounds

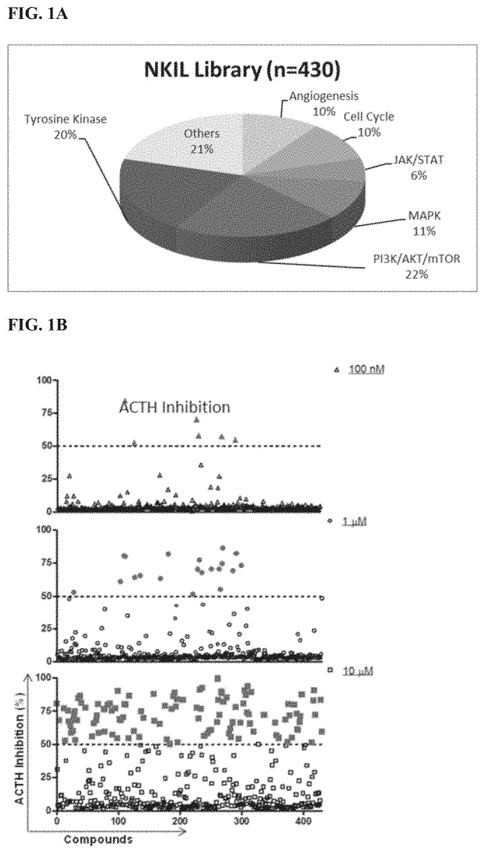

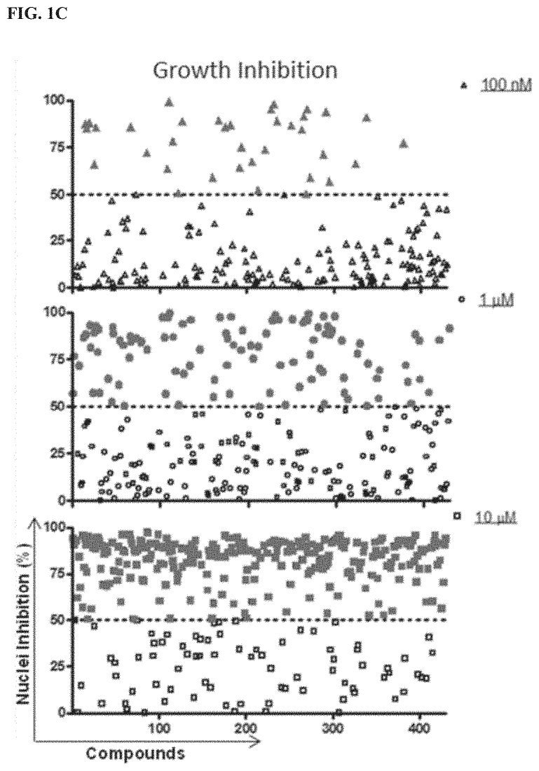

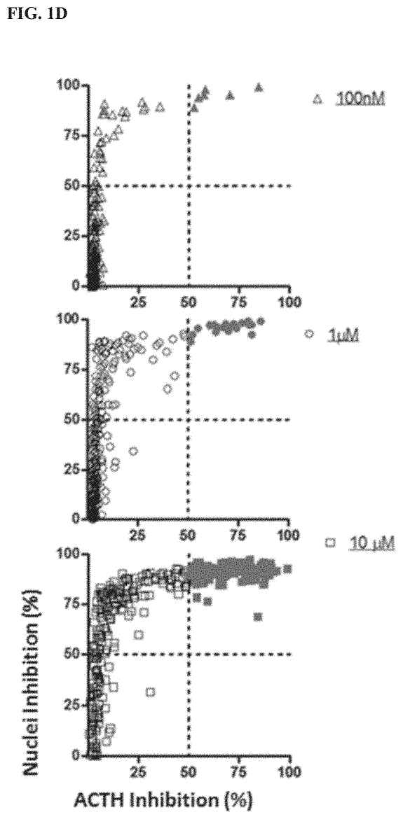

Using ACTH AlphaLISA assay described herein in combination with nuclei staining (Hoechst 33342 dye), the inventors screened an annotated kinase inhibitor library (KIL, n=430) at 100 nM, 1 μM and 10 μM. The KIL contained inhibitors of PI3K/AKT/mTOR (n=95), protein tyrosine kinases (n=87), MAPK (n=45), angiogenesis (n=44), cell cycle (n=44), JAK/STAT (n=26), and others (n=89, FIG. 1A). Out of 430 compounds screened, 6, 20 and 115 compounds exhibited >50% ACTH AlphaLiSA inhibition (FIG. 1B), and 36, 105 and 263 compounds exhibited >50% nuclei inhibition (FIG. 1C) at doses of 100 nM, 1 μM and 10 μM respectively. Among the 6 compounds that exhibited efficacy at 100 nM (FIG. 1D), PI3K/HDAC inhibitor CUDC-907, PI3K/AKT/mTOR inhibitor BGT226 and PLK inhibitor BI-2536 are being studied in Phase II clinical trials.

### Example 3: Activity of Exemplary Compounds

Table 1 depicts the activity of certain exemplary compounds described herein against ACTH production.

TABLE 1

| | ACTH Inhibition (%) | | | Nuclei Inhibition (%) | | |
|---|---|---|---|---|---|---|
| | 100 | 1 | 10 | 100 | 1 | 10 |
| Compound | nM | μM | μM | μM | μM | μM |
| CUDC-907 | 84.7 | 80.1 | 80.0 | 90.1 | 99.2 | 99.3 |
| PF-3758309 | 70.4 | 70.2 | 77.5 | 91.5 | 95.6 | 95.2 |
| Dinaciclib (SCH727965) | 58.1 | 77.3 | 69.2 | 92.0 | 98.3 | 98.0 |
| BGT226 (NVP-BGT226) | 57.5 | 86.3 | 88.2 | 94.3 | 99.0 | 95.4 |
| BI 2536 | 54.9 | 82.0 | 77.7 | 93.4 | 92.3 | 94.0 |
| PHA-793887 | 52.5 | 64.3 | 59.9 | 88.3 | 93.7 | 88.9 |

### Example 4: Comparison of CUDC-907 with Certain HDAC and PI3K Inhibitors

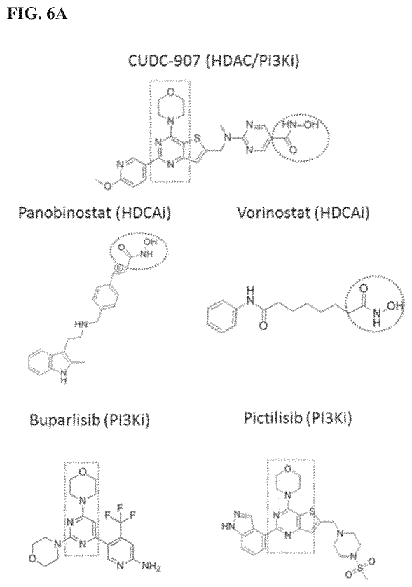

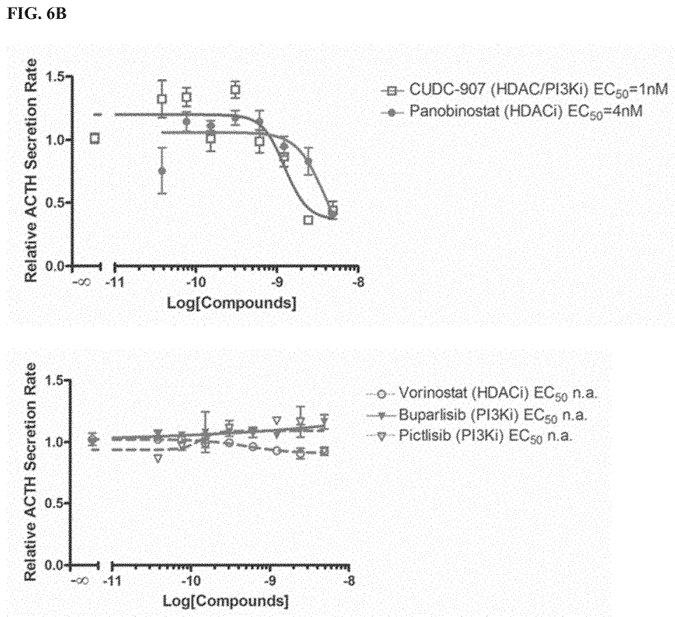

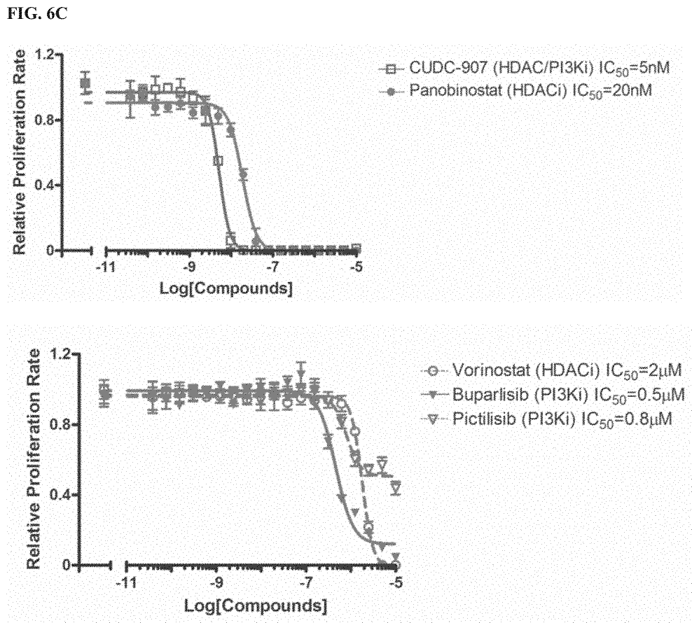

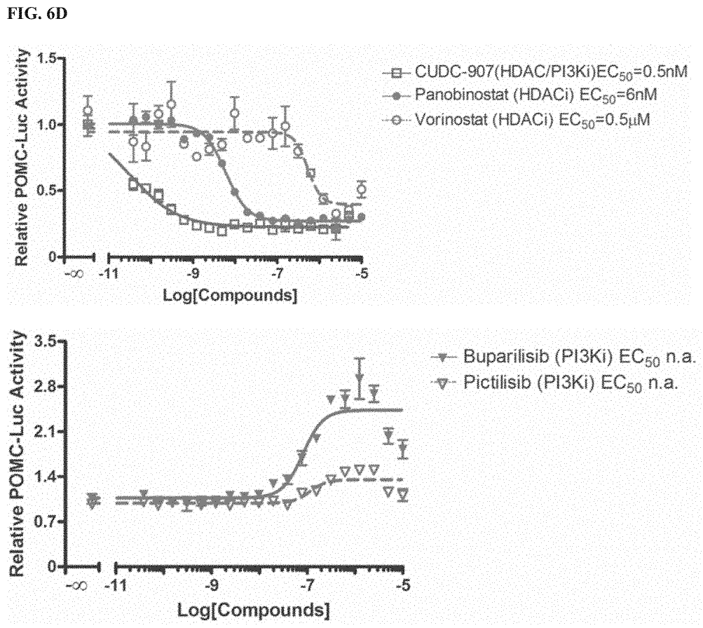

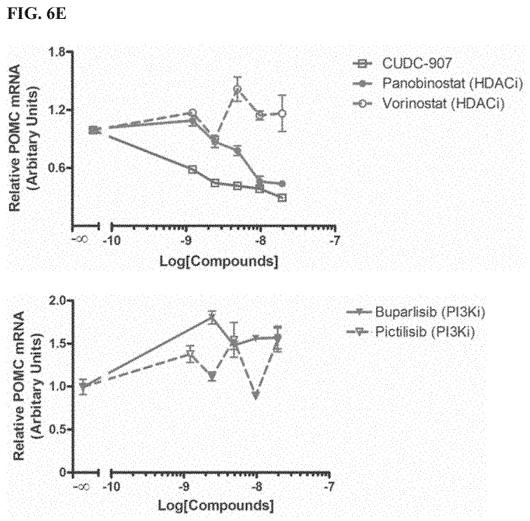

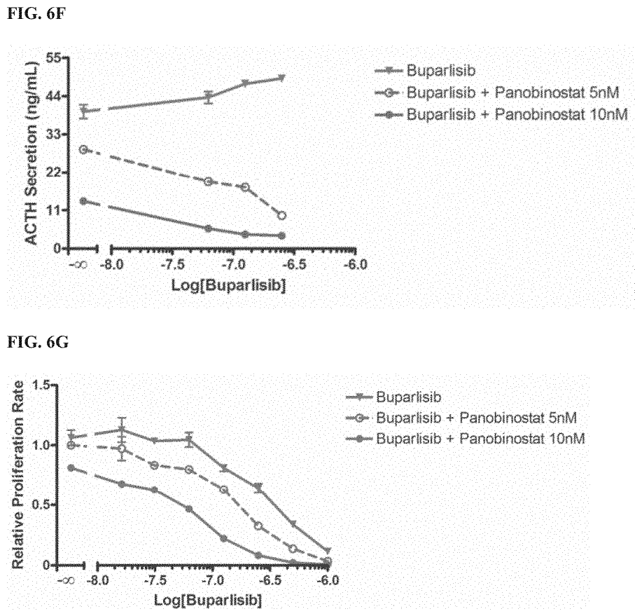

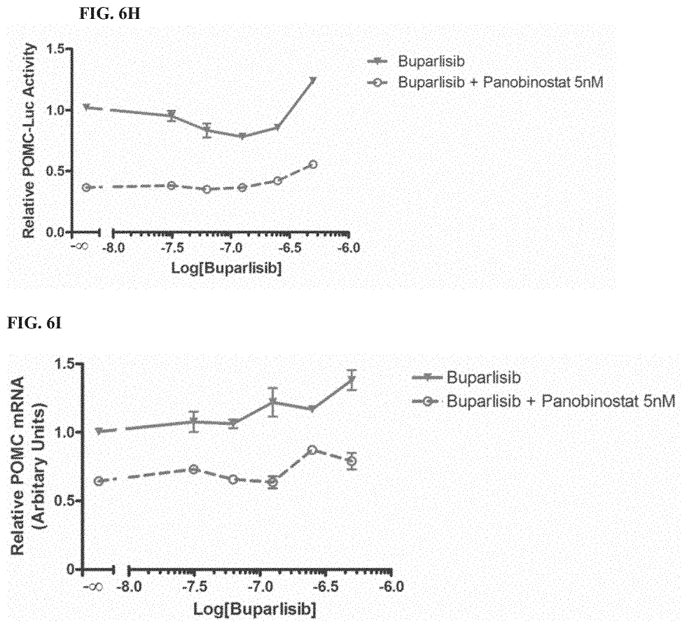

CUDC-907 was synthesized by integration of a HDAC inhibitory functional moiety into a core PI3K inhibitor structure scaffold (FIG. 6A). To better understand the contribution of HDAC versus PI3K inhibitory activities of CUDC-907 in suppressing corticotroph tumor ACTH secretion and proliferation, the actions of CUDC-907 with the single-target HDAC inhibitors panobinostat and vorinostat, and the single-target PI3K inhibitors buparlisib and pictilisib was compared. CUDC-907 and panobinostat potently inhibited ACTH secretion with EC₅₀ of 1 nM and 4 nM respectively (FIG. 6B, upper panel), compared to another HDACi (vorinostat) or the PI3Kis (buparlisib and pictilisib) at the doses tested (0.4-40 nM, FIG. 6B lower panel). Similarly, CUDC-907 and panobinostat exhibited comparable inhibitory effects on AtT20 cell proliferation with IC₅₀ of 5 and 20 nM respectively (FIG. 6C, upper panel), which were more potent than those observed for the HDAC inhibitor vorinostat (IC₅₀ 2 μM), and the PI3K inhibitors buparilisib (IC₅₀ 0.5 μM) and pictilisib (IC₅₀ 0.8 μM, FIG. 6C, lower panel).

Using a POMC promoter-driven luciferase assay, the direct actions of the aforementioned compounds on POMC transcription was examined. CUDC-907 and panobinostat and vorinostat inhibited POMC transcription with a range of potencies, (CUDC-907 EC₅₀ 0.5 nM to vorinostat 0.5 μM) (FIG. 6D, upper panel). In contrast, buparlisib and pictilisib increased POMC transcription at higher doses (FIG. 6D lower panel). Quantitation of POMC mRNA expression by RT-PCR showed that only CUDC-907 resulted in a potent reduction in POMC mRNA expression as compared to the other HDAC inhibitors (FIG. 6E, upper panel), and the two PI3K inhibitors did not inhibit POMC mRNA expression (FIG., lower panel).

To further explore whether the PI3K inhibitory action synergized with the HDACi-mediated downregulation of ACTH secretion, the effects of combination of single agent non-selective HDAC inhibitor panobinostat and the PI3K inhibitor buparlisib were investigated. As shown in FIG. 6F, buparlisib alone did not inhibit ACTH secretion. In contrast, panobinostat (5 and 10 nM) inhibited ACTH secretion by up to 66% (ACTH secretion (ng/mL), Veh: 39.6±2 vs. Panobinostat 5 nM: 28.7±0.1, p<0.05; Panobinostat 10 nM: 13.6±1, p<0.01, FIG. 6F). Combination treatment of panobinostat (5 and 10 nM) with buparlisib (62.5 nM) further inhibited ACTH secretion by up to 85% respectively (ACTH secretion (ng/mL), Buparlisib+Panobinostat 5 nM: 19.3±0.1, p<0.005; Buparlisib+Panobinostat 10 nM: 5.8±1, p<0.01, FIG. 6F). Panobinostat was not as potent an inhibitor of corticotroph tumor proliferation compared to CUDC-907 (IC₅₀ of panobinostat 20 nM vs. CUDC-907 5 nM) (FIG. 6C) and, as shown in FIG. 6G, panobinostat alone (5 and 10 nM) marginally inhibited murine corticotroph proliferation as compared to vehicle (Relative Proliferation Rate, Veh: 1.0±0.06 vs. Panobinostat 5 nM: 1.0±0.01, n.s.; Panobinostat 10 nM: 0.8±0.005, n.s., FIG. 6G). However, addition of buparlisib increased inhibition of corticotroph tumor proliferation, while buparlisib alone did not affect cell proliferation (Relative Proliferation Rate, buparlisib 62.5 nM: 1.0±0.06; Buparlisib+Panobinostat 5 nM: 0.8±0.03, n.s.; Buparlisib+Panobinostat 10 nM: 0.5±0.01, p<0.05; FIG. 6G) Inhibition of POMC transcription (FIG. 6H) and mRNA expression (FIG. 6I) were not further increased by addition of buparlisib. Taken together, these results demonstrate that CUDC-907 exerts much of its inhibitory effect on ACTH secretion by its HDAC inhibitory activity to reduce POMC transcription, while PI3K-mediated inhibition of corticotroph tumor cell viability further contributes to reduced ACTH secretion. In words, CUDC-907 is a promising candidate for the treatment of Cushing's disease due to its ability to inhibit ACTH secretion and PI3K.

### Example 5: Involvement of Nuclear Receptors in CUDC-907-Inhibition of POMC Transcription

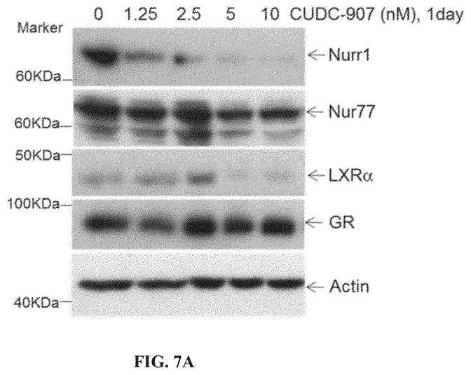

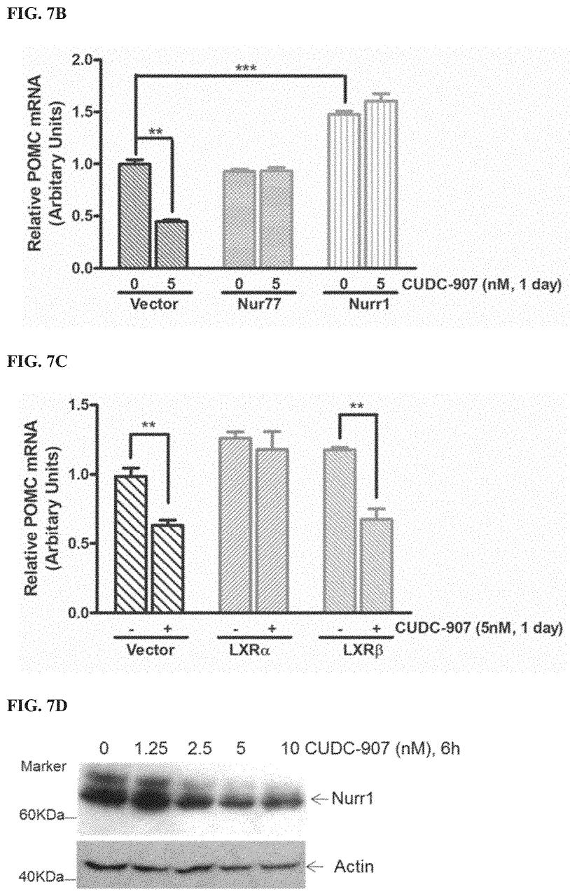

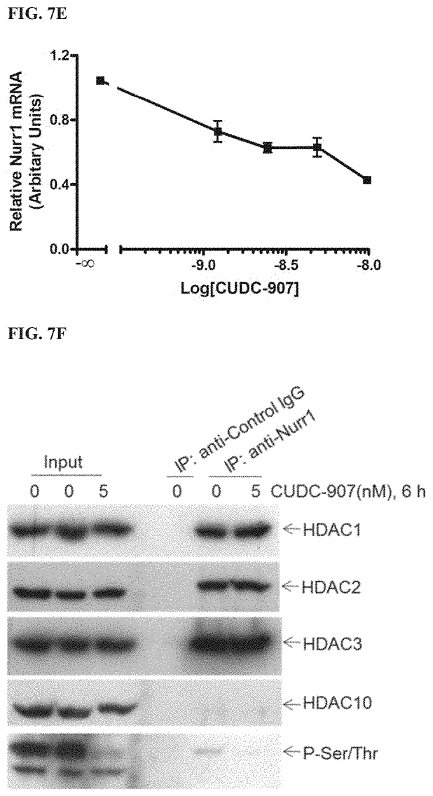

CUDC-907 potently inhibits HDAC classes I (IC₅₀ of 1.7, 5.0, and 1.8 nM for HDAC1, 2 and 3) and II enzymes (IC₅₀ of 2.8 nM for HDAC10). However, because, HDACs do not contain canonical DNA-binding domains, and are recruited to chromatin by protein-protein interactions with other DNA-associated factors, it was critical to characterize the molecular partners of HDACs involved in CUDC-907's regulation of POMC transcription. Accordingly, the expressions of several nuclear receptors, known as POMC positive and negative regulators, including Nurr1 (NR4A2), Nur77 (NR4A1), LXRα (NR1H3), and GR (NR3C1), were examined. Treatment of CUDC-907 at low dose (1.25 nM, 24 h) readily led to reduction in Nurr1 expression (FIG. 7A). Additionally, reductions in Nur77 and LXRα expression was observed following the administration of higher concentrations of CUDC-907 (5 and 10 nM, 24 h, FIG. 7A. Additionally, minor effects on the expression of GR (FIG. 7A) was observed.

To determine the involvement of these nuclear factors in CUDC-907-mediated POMC inhibition, studies wherein Nur77, Nurr1, LXRα, and LXRβ were overexpressed were performed. These studies demonstrated that overexpression of Nur77 and Nurr1 blocked CUDC-907 inhibition of POMC mRNA expression (Relative POMC mRNA, Veh vs. CUDC-907 5 nM, Vector, 1.0±0.02 vs. 0.4±0.01 p<0.01; Nur77, 1.2±0.02 vs. 1.2±0.02 n.s.; Nurr1, 1.5±0.07 vs. 1.4±0.05 n.s., FIG. 7B). Nurr1 also potently increased basal POMC mRNA expression (Relative POMC mRNA, Vector vs. Nurr1 1.0±0.02 vs. 1.5 f 0.07 p<0.005, FIG. 7B). Overexpression of LXRα but not LXRβ also blunted the effect of CUDC-907 on POMC inhibition (Relative POMC mRNA, Veh vs. CUDC-907 5 nM, Vector, 1.0±0.03 vs. 0.6±0.02 p<0.01; LXRα 1.3±0.03 vs. 1.2±0.07 n.s.; LXRβ, 1.2±0.01 vs. 0.7±0.04 p<0.01, FIG. 7C), indicating LXRα and not LXRβ, may be responsible for CUDC-907 efficacy. The effect of CUDC-907 on Nurr1 downregulation was then examined and it was observed that the 6 h-treatment of CUDC-907 led to dramatic inhibition of Nurr1 protein expression (FIG. 7D), this observation was consistent with inhibition of Nurr1 mRNA expression (FIG. 7E).

Additionally, it was observed that Nurr1 interaction with HDAC-1, 2, 3, but not HDAC10, was unaffected by CUDC-907 (FIG. 7F). However, Nurr1 serine/threonine phosphorylation levels were dramatically inhibited by CUDC-907 (FIG. 7F); this may have occurred due to CUDC-907's effect on PI3K pathway. Taken together, these findings suggest that CUDC-907 inhibits POMC transcription in part through regulating nuclear factors, such as Nur and LXRα. In short, CUDC-907 not only downregulated Nurr1 expression, but, also inhibited its phosphorylation.

### Example 6: CUDC-907 Increased Expression of Cell Cycle Inhibitors and Induced Apoptosis

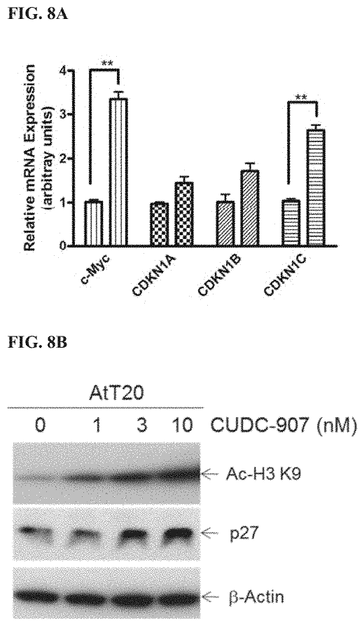

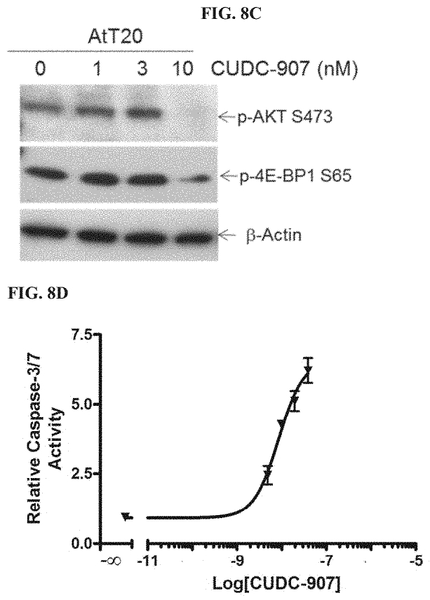

c-Myc has been reported to mediate the inhibitory effect of CUDC-907 on cell proliferation in several Myc-dependent cancers. However, Myc has not been demonstrated to be a contributing factor in the proliferation of pituitary tumors. Interestingly, it was observed that CUDC-907 increased c-Myc mRNA expression in murine corticotroph tumor cells (FIG. 8A) and, further, the overexpression of c-Myc did not affect CUDC-907-mediated inhibition of corticotroph tumor proliferation. However, CUDC-907 did increase expression of several cell cycle inhibitors, particularly CDKN1C which encodes p57 (Relative CDKN1C mRNA expression, Veh 1.0±0.06 vs. CUDC-907 2.6±0.1, p<0.01) concomitant with elevated histone acetylation (Ac-H3-K9, FIG. 8B). Further, given its PI3K inhibitory activity, CUDC-907 treatment also blocked AKT activation and its downstream target 4E-BPI (FIG. 8C), therefore increasing the activity of apoptosis executors caspase-3 and -7 (FIG. 8D). In totality, these findings demonstrated the multiple synergistic actions of CUDC-907 inhibit corticotroph tumor proliferation through both HDACi-mediated cell cycle arrest and PI3Ki-mediated promotion of corticotroph tumor apoptosis.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating a disease or disorder characterized by an increased secretion of adrenocorticotropic hormone, comprising administering to a subject in need thereof a compound that inhibits both the secretion of adrenocorticotropic hormone and tumor growth.

2. The method of claim 1, wherein the disease or disorder is Cushing's disease.

3. The method of claim 1, wherein the disease or disorder is hypercortisolism, Itsenko-Cushing syndrome, hyperadrenocorticism, or Cushing's Syndrome.

4. A method of treating Cushing's disease, comprising administering to a subject in need thereof a compound that inhibits both the secretion of adrenocorticotropic hormone and tumor growth.

5. The method of claim 1, wherein the compound is a PI3K inhibitor, a HDAC inhibitor, a PKA inhibitor, a CDK inhibitor, a AKT inhibitor, a mTOR inhibitor, a PLK inhibitor, a cell cycle inhibitor, or an inhibitor of cytoskeletal signaling.

6. The method of claim 1, wherein the compound is a PI3K inhibitor, a HDAC inhibitor, a PKA inhibitor, a CDK inhibitor, a AKT inhibitor, a mTOR inhibitor, or a PLK inhibitor.

7. The method of claim 1, wherein the compound is a PI3K inhibitor.

8. The method of claim 1, wherein the compound is an HDAC inhibitor.

9. The method of claim 1, wherein the compound is a PKA inhibitor.

10. The method of claim 1, wherein the compound is a CDK inhibitor.

11. The method of claim 1, wherein the compound is an AKT inhibitor.

12. The method of claim 1, wherein the compound is a mTOR inhibitor.

13. The method of claim 1, wherein the compound is a PLK inhibitor.

14. The method of claim 1, wherein the compound is 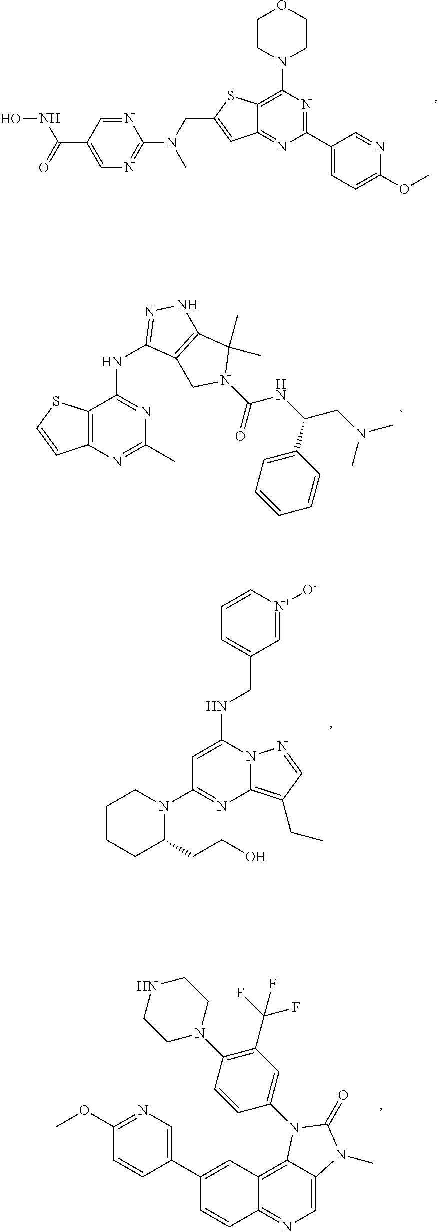 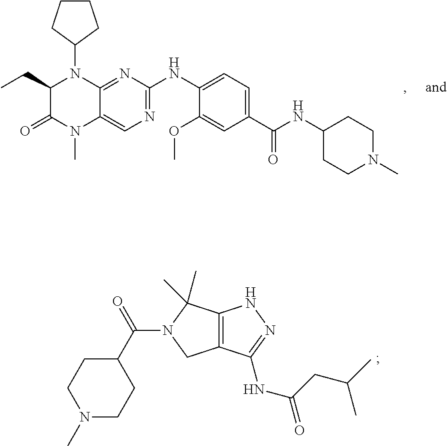 [SMILES: CC[C@@H]1C(=O)N(C)c2cnc(Nc3ccc(C(=O)NC4CCN(C)CC4)cc3OC)nc2N1C1CCCC1 ; CC(C)CC(=O)Nc1n[nH]c2c1CN(C(=O)C1CCN(C)CC1)C2(C)C]
or a pharmaceutically acceptable salt thereof.

15-19. (canceled)

20. The method of claim 1, wherein the method further comprises administering at least one additional compound.

21-23. (canceled)

24. The method of claim 1, wherein the additional compound is a PI3K inhibitor, a HDAC inhibitor, a PKA inhibitor, a CDK inhibitor, a AKT inhibitor, a mTOR inhibitor, a PLK inhibitor, a cell cycle inhibitor, or an inhibitor of cytoskeletal signaling.

25. The method of claim 20, wherein the additional compound is 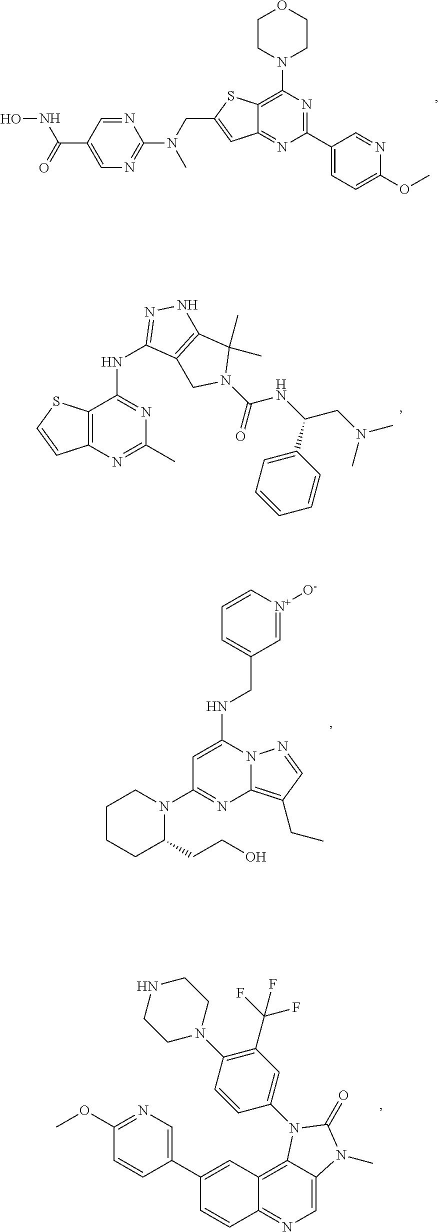 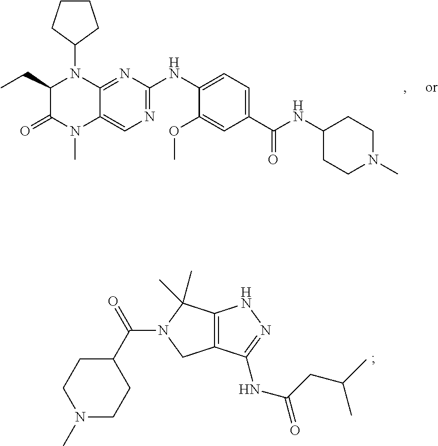 [SMILES: CC[C@@H]1C(=O)N(C)c2cnc(Nc3ccc(C(=O)NC4CCN(C)CC4)cc3OC)nc2N1C1CCCC1 ; CC(C)CC(=O)Nc1n[nH]c2c1CN(C(=O)C1CCN(C)CC1)C2(C)C]
or a pharmaceutically acceptable salt thereof.

26-30. (canceled)

31. The method of claim 1, wherein the method is performed continuously for at least 12 months.

32. The method of claim 1, wherein the method is performed continuously for at least 24 months.

33. A method of identifying a compound that inhibits the secretion of adrenocorticotropic hormone (ACTH) and tumor growth, comprising the steps of:
a) contacting a plurality of AtT20 cells with the compound, thereby forming an assay mixture;
b) quantifying the ability of the compound to inhibit the secretion of ACTH; and
c) quantifying the ability of the compound to inhibit nuclei;
wherein the compound is identified as being an inhibitor of ACTH secretion and tumor growth if the compound inhibits both ACTH secretion and nuclei growth at a predetermined threshold.

34-65. (canceled)
